(19) United States
(12) Patent Application Publication
(10) Pub. No.: US 2022/0033748 A1
(43) Pub. Date: Feb. 3, 2022

(54) MULTICOMPARTMENT BAG FOR CULTIVATION OF CELLS

(71) Applicant: GLOBAL LIFE SCIENCES SOLUTIONS USA LLC, MARLBOROUGH, MA (US)

(72) Inventors: MATTHEW DAVID OUELLETTE, NEWTON, MA (US); MICHAEL JASON MILLER, SHREWSBURY, MA (US)

(73) Assignee: GLOBAL LIFE SCIENCES SOLUTIONS USA LLC, MARLBOROUGH, MA (US)

(21) Appl. No.: 17/278,718

(22) PCT Filed: Sep. 12, 2019
(86) PCT No.: PCT/EP2019/074365

§ 371 Date: Mar. 23, 2021

Related U.S. Application Data
(60) Provisional application No. 62/737,309, filed on Sep. 27, 2018.

(51) Int. Cl. C12M 1/00; C12M 3/06; C12M 1/34

(52) U.S. Cl. CPC C12M 23/14; C12M 23/26; C12M 41/44; C12M 23/16; C12M 23/34

(57) ABSTRACT

The invention discloses a flexible plastic bag for cultivation of cells, including a top wall film and a bottom wall film, each having an inside and an outside, sealed to each other inside-to-inside by durable weld seams, optionally via one or more side wall films, to form a bag with an inner volume delimited by the durable weld seams; one or more ports through the top and/or bottom wall film for introduction and withdrawal of fluids; one or more frangible weld seams joining the insides of said top and bottom wall films, dividing the bag inner volume into a plurality of cultivation compartments; and one or more gripping means affixed to the top and bottom wall films, adjacent each of the frangible weld seams and adapted to break a specific frangible weld seam by pulling apart the gripping means on the top and bottom wall films.

### CROSS REFERENCE TO RELATED APPLICATIONS

This application is a national stage of International Application No. PCT/EP2019/074365 filed on Sep. 12, 2019, which claims priority to U.S. Provisional Application No. 62/737,309 filed on Sep. 27, 2018, all of which are hereby incorporated by reference in their entireties.

### TECHNICAL FIELD OF THE INVENTION

The present invention relates to bioreactors for cell cultivation, and more particularly to flexible bag bioreactors suitable for multi-stage expansion of cell cultures, such as seed train expansion or expansion of cells for cell therapy. The invention also relates to a method of expanding cell cultures in flexible bag bioreactors.

### BACKGROUND OF THE INVENTION

When cell cultures are scaled up from a small cell bank sample to larger production batches, this normally has to be made in several steps, using separate bioreactors. This sequence of cultivations is often called a seed train and is needed in order to keep the cell densities within a certain optimal window, often in the order of >10⁵ cells per ml. In large scale production of biopharmaceuticals where the scale-up is from a small cryopreserved vial sample up to several m³, the seed train can involve up to six steps and take several weeks. It is also a complex procedure in that sterile transfer of the culture from one bioreactor to another is needed, such that transfers have to be made in LAF benches or sterile cleanrooms. Even under these conditions there is also a certain risk for adventitious infections, which can have disastrous consequences for a high value large scale culture. Similar concerns apply also for expansion of cells, e.g. stem cells or engineered immune cells, to be used in clinical cell therapy.

With the general trend towards single-use vessels for cell culture, there is an increasing tendency towards using flexible bag bioreactors in seed trains. However, the need to empty one smaller bag and to transfer the content into a larger bag still remains and is a work-intensive operation with some risk of contamination. It has been suggested to gradually increase the culture volume in flexible bags either by clamping off part of the bag over the entire bag cross section and then removing the clamp (WO2008153401) or by starting with a folded bag and then unfolding it ((US20100055764). However, these solutions do not provide good sealing between the used and unused compartments, leading to leakage of culture into the unused compartments and contamination of the cell culture by substances released from cells grown under unsuitable conditions. These methods also involve a substantial risk of mechanical damage to the bags, with resulting risks of bag rupture.

Accordingly, there is a need for a safe and convenient way of transferring cell cultures from one flexible culture compartment to another under sterile conditions. There is further a need for automation of the seed train procedures.

### SUMMARY OF THE INVENTION

One aspect of the invention is to provide a flexible bag which allows convenient increase of the culture volume without any open transfer of the culture between vessels, thus mitigating the risk of contamination. This is achieved with a flexible plastic bag for cultivation of cells, including:

a top wall film and a bottom wall film, each having an inside and an outside, sealed to each other inside-to-inside by durable weld seams, optionally via one or more side wall films, to form a bag with an inner volume delimited by the durable weld seams;

one or more ports through the top and/or bottom wall film(s) for introduction and withdrawal of fluids;

one or more frangible weld seams joining the insides of the top and bottom wall films, dividing the inner volume into a plurality of cultivation compartments; and

one or more gripping means affixed to the top and bottom wall films, adjacent each of the frangible weld seams and adapted to break a specific frangible weld seam by pulling apart the gripping means on the top and bottom wall films adjacent the specific frangible weld seam.

One advantage is that the seams between the cultivation compartments are easily broken to form a larger cultivation compartment. Further advantages are that the breaking of the seams can be automated, that operator time can be minimized and that the requirements for bags and equipment can be decreased.

A second aspect of the invention is to provide a bioreactor with one or more flexible bags as discussed above on a moving (e.g. rocking) platform to provide agitation, which allows convenient increase of the culture volume without any open transfer of the culture between vessels. This is achieved with a bioreactor as defined in the claims.

A third aspect of the invention is to provide a cultivation method where the culture volume is increased without any open transfer of the culture between vessels. This is achieved with a method as defined in the claims.

Further suitable embodiments of the invention are described in the dependent claims.

### DRAWINGS

FIG. 1 shows an embodiment of the invention with a bag having three connectable cultivation compartments delimited from each other by two frangible weld seams (top view). The bag is mounted on a rocking table.

FIG. 2 shows a series of side views for the bag of FIG. 1. a) both frangible weld seams intact, b) one frangible weld seam opened and c) both frangible weld seams opened.

FIG. 3 shows alternative arrangements of the bag (top views), with a) diagonal frangible weld seams and b) concentric frangible weld seams.

FIG. 4 shows a film loop gripping means in detail (side view across a frangible weld seam).

FIG. 5 shows a film tab gripping means in detail (side view across a frangible weld seam).

FIG. 6 shows an adhesive tape gripping means in detail (side view across a frangible weld seam).

FIG. 7 shows a port bridge gripping means in detail (side view across a frangible weld seam).

FIG. 8 shows a single port gripping means in detail (side view across a frangible weld seam).

FIG. 9 shows a gripping handle in detail (side view across a frangible weld seam).

FIG. 10 shows a film fold gripping means in detail (side view across a frangible weld seam).

FIG. 11 shows two seam opener embodiments for opening of the frangible weld seams (side views).

FIG. 12 shows a rocking table bioreactor with a bag (side view).

FIG. 13 shows a schematic side view along a frangible weld seam.

### DEFINITIONS

The term “durable weld seam”, as used herein means a weld seam joining two plastic films or laminates, which is not possible to pull apart without damage to the films or which needs a force of more than about 80 N to pull apart.

The term “frangible weld seam”, as used herein means a weld seam joining two plastic films or laminates which can be pulled apart without damaging the films, using a moderate force, such as about 5 N to about 80 N. Such seams are also capable of withstanding the forces occurring during normal cell cultivation without rupturing or causing premature leakage.

The term “port” as used herein means an opening in a bag for transport of fluid into and/or out of the bag. The port typically includes an internal fitting (e.g. a disc) for attachment to a bag film and an external fitting for attachment to tubing or an external device. The external fitting can e.g. be a hose barb or other tubing connector, a length of tubing, a membrane for piercing by a spike or syringe needle etc.

### DETAILED DESCRIPTION OF EMBODIMENTS

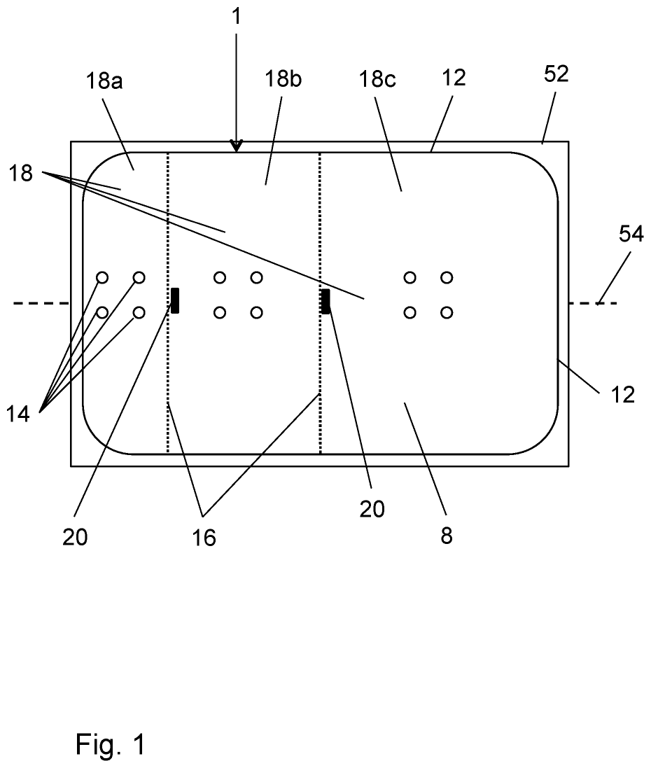

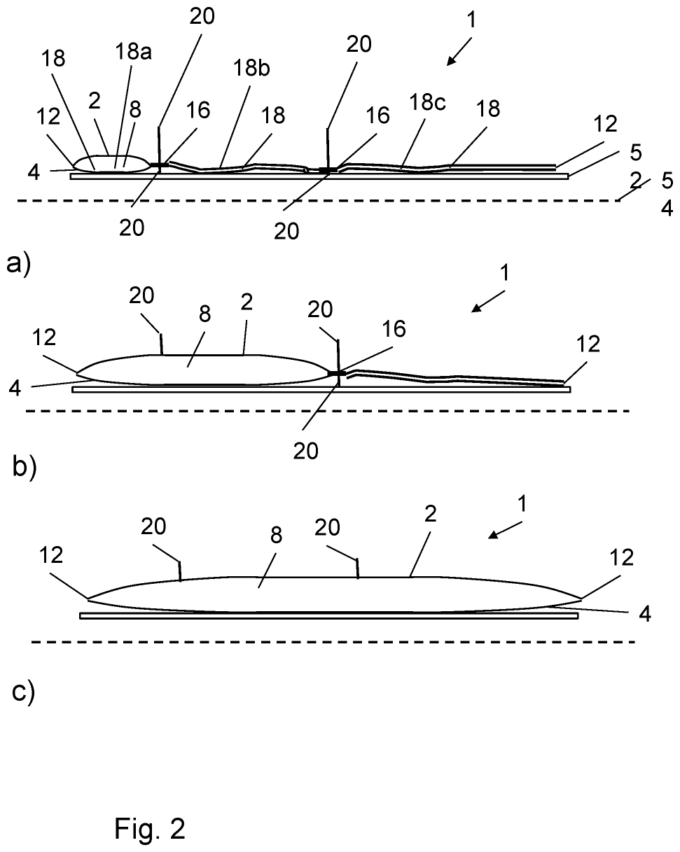

In one aspect, illustrated by FIGS. 1-10, the present invention discloses a flexible plastic bag 1 for cultivation of cells. The bag including:

a) A top wall film 2 and a bottom wall film 4, with each film having an inside (facing an inner volume 8 of the bag) and an outside (facing the exterior). These films are sealed to each other inside-to-inside by durable weld seams 12 to form a bag with an inner volume where the inner volume 8 faces the insides of both the top and bottom wall films and is delimited by the durable weld seams and the top and bottom wall films. The durable weld seams can suitably be located along the edges of the top and bottom wall films. The top and bottom wall films can preferably be welded directly to each other, but they may also be sealed via one or more side wall films, to form a bag with an inner volume. The films may be homogeneous films or laminates and may include polyolefins, such as e.g. polyethylenes and/or ethylene vinyl acetate copolymers, but also barrier layers, e.g. ethylene vinyl alcohol polymers and/or tear resistant layers of e.g. polyamides. The thickness of the films/laminates may e.g. be 50-400 micrometers, such as 100-350 micrometers. Suitably, the film/laminate materials can be of USP VI quality, with low levels of leachables/extractables and may be selected for their suitability in cell cultivation applications. Examples of such films/laminates for cell cultivation include the Fortem™, Bioclear 10 and Bioclear 11 laminates from GE Healthcare Life Sciences.

b) One or more ports 14 through the top and/or bottom wall film for introduction and withdrawal of fluids. At least one cultivation compartment, as discussed below under c), suitably includes a gas inlet and a gas outlet. These inlets and outlets may be equipped with sterile filters (not shown) to prevent infection/contamination of the culture and are used to supply e.g. air/oxygen to the culture and to remove gaseous metabolites, such as carbon dioxide. The cultivation compartment(s) may also include one or more of sampling outlets, inlets for culture medium and sensors for e.g. temperature, cell density, pH and/or concentrations of e.g. oxygen or metabolites. At least a first cultivation compartment should have the port(s)/sensor(s) required for a particular cultivation protocol. These will then be accessible also when further cultivation compartments are added to the first. However, when the compartment volume increases above a certain level it can be advantageous to have further ports, e.g. with larger tubing diameters, to accommodate for the higher flow rates needed at larger scale.

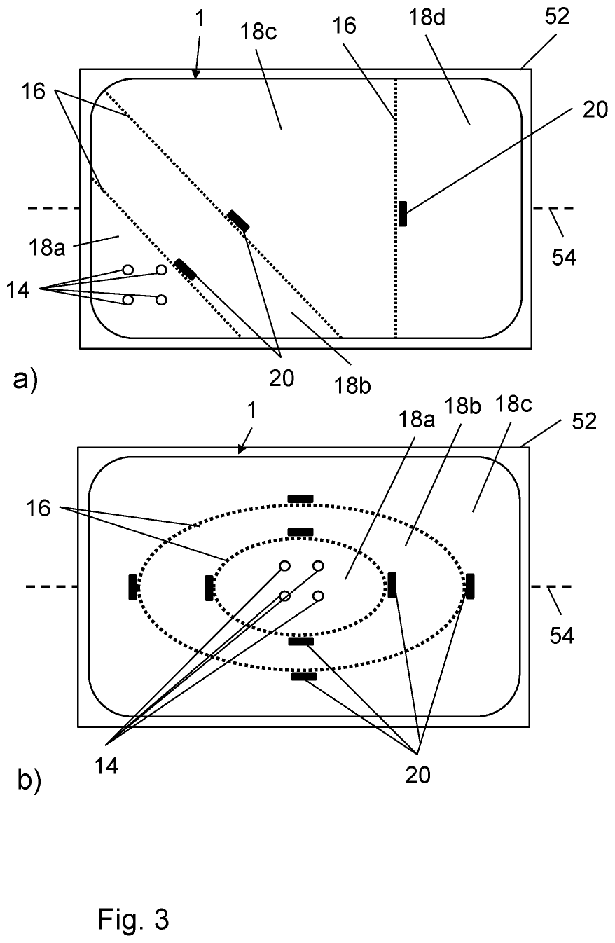

c) One or more frangible weld seams 16 joining the insides of the top and bottom wall films, dividing the inner volume into a plurality of cultivation compartments 18, such as at least three cultivation compartments. Each cultivation compartment is then delimited by the top and bottom wall films, one or more frangible weld seams and, optionally, one or more durable weld seams. During use, the bag may include a cell culture in at least one of the cultivation compartments and, when unbroken, the frangible weld seams prevent any leakage of culture into another cultivation compartment. The frangible weld seams may e.g. be weak welds, as disclosed in EP 2,226,058A1 or U.S. Pat. No. 4,519,499, which are hereby incorporated by reference in their entireties. A frangible weld seam can e.g. constitute the entire delimitation between two adjacent cultivation compartments. As shown in FIGS. 1 and 3 a), the frangible weld seams may extend between two durable weld seams, but they can also form closed loops as in FIG. 3 b). The cultivation compartments can be a first 18a, a second 18b and optionally a third 18c and yet optionally a fourth 18d cultivation compartment. The second cultivation compartment may be larger than the first cultivation compartment, e.g. having a volume of at least 120%, such as at least 200%, 120-1000% or 250-300% of the volume of the first cultivation compartment. The third cultivation compartment may be larger than the second cultivation compartment, e.g. having a volume of at least 120%, such as at least 200%, 120-1000% or 250-300% of the volume of the second cultivation compartment, and/or at least 140%, such as at least 400% or 140-10 000% of the volume of the first cultivation compartment. This allows a safe and convenient three-step scale-up from the first, to the second and then to the third cultivation compartment. If all the frangible weld seams are broken, the entire inner volume of the bag will form a single cultivation compartment, which is then delimited by the top and bottom films and the durable weld seams. The arrangement of the cultivation compartments can e.g. be linear as in FIG. 1, diagonal as in FIG. 3 a) or concentric as in FIG. 3 b), although other arrangements are also possible. The bag may also include further cultivation compartments, e.g. with a total of four, five or more cultivation compartments delimited by frangible weld seams. As an example, a bag may include five cultivation compartments with volumes of 200 ml, 500 ml, 1.5 l, 5 l and 15 l respectively. Further, the bag may include one or more cultivation media compartments, delimited by frangible weld seams. In this case, the frangible weld seams can be opened to allow influx of fresh culture medium from a cultivation media compartment into a cultivation compartment when needed. The cultivation media compartment(s) may be pre-filled with cultivation medium upon delivery of the bag, or they may be filled with the appropriate cultivation medium by the operator.

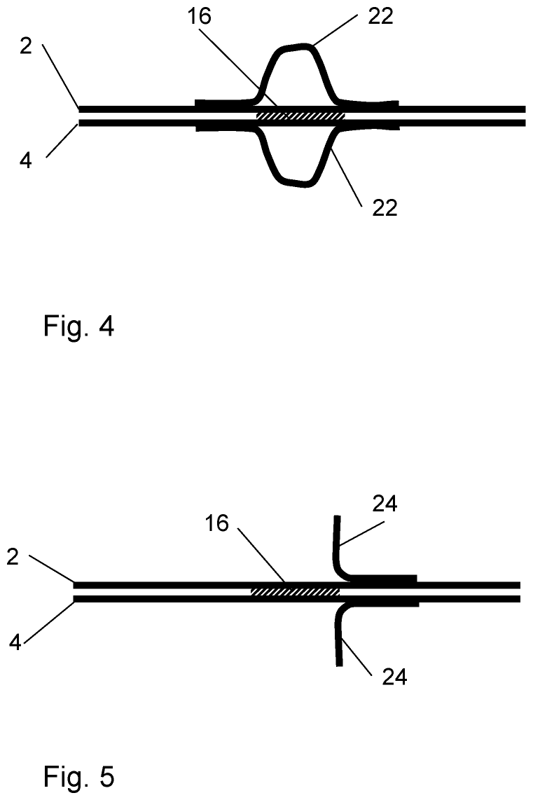

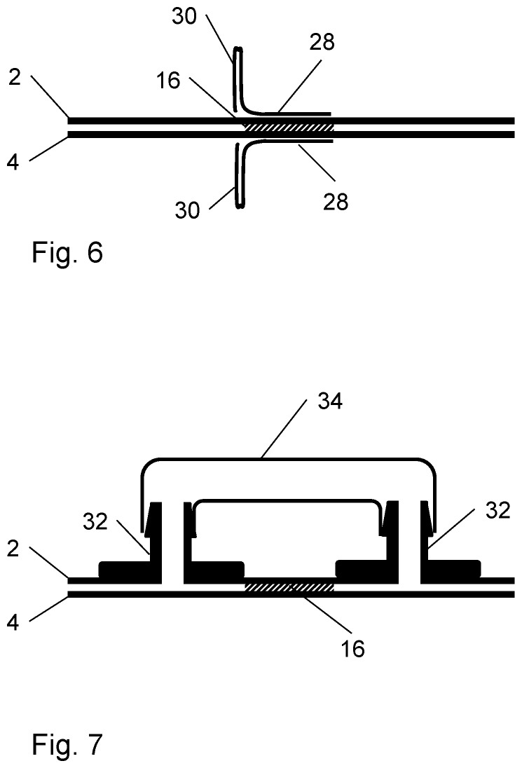

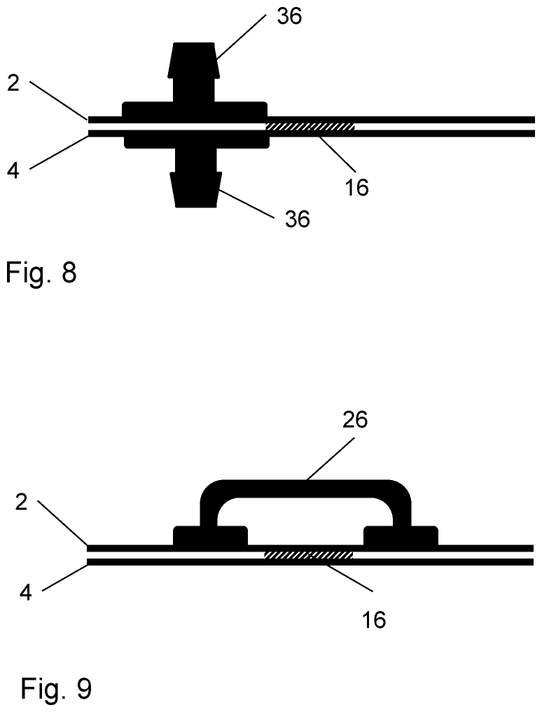

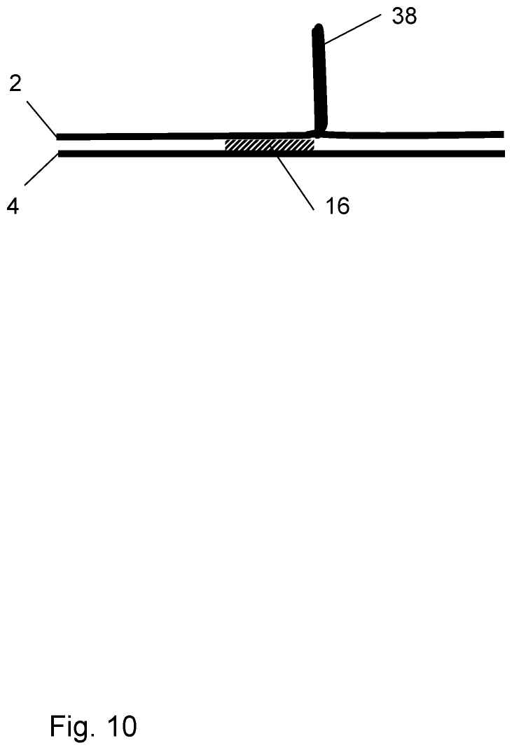

d) One or more gripping means 20 affixed to the outsides of the top and bottom wall films (or on the insides, with part of the gripping means protruding through an aperture in the film to be grippable from the outside), adjacent each of the frangible weld seams and adapted to break a specific frangible weld seam by pulling apart the gripping means on the top and bottom wall films adjacent the specific frangible weld seam. The gripping means may include one or more of: a loop of film 22, a tab of film 24, a hook, a gripping bar, a handle 26, a strip of adhesive tape 28 with a tab 30 or loop, a pair of ports 32 connected with a tube 34, a single port 36 and a fold 38 of the top/bottom film. The gripping means can be arranged pairwise on locations in the top and bottom films facing each other. Further details of these embodiments are discussed below:
Loops of film 22 sealed into the bag outer surface by welding, so that the user can grip underneath the loops and pull (FIG. 4).
Film sealed into bag outer surface by welding (FIG. 5). This is similar to the above embodiment, but not necessarily in the form of loops. A simple pull tab 24 sealed into the film or forming part of the film can suffice. The tab can also have one or more holes punched through the tab/film for attachment of a hook or a similar component of a mechanical seam opener.
Instead of sealing in pull tabs, adhesive tape 28 or similar can be used to create a grabbable surface 30 near the frangible seam (FIG. 6). Apply a strip of tape above the frangible seam and the user can pull on the tape itself to open the seam.
Similar to the tape, a gripping handle 26 can be attached to the bag near the frangible seam to provide the correct means of peeling the frangible seam (FIG. 9). This handle can be attached either by welding or by adhesive bonding.
A “port bridge” can be created by welding ports 32 (e.g. barbed ports) near each side of the frangible seam (FIG. 7). The two ports can be connected by a tube 34 to create a handle that spans the frangible seam.
Similar to the port bridge, but if one port 36 (e.g. a blind port without any opening or with a blocked opening) is welded into the film near the frangible seam it can be gripped (e.g. with tubing or a handle/hook structure on the port) so that the seam can be peeled (FIG. 8).
Intentionally create “excess” film that can be pinched by user when manufacturing the bag (FIG. 10). For instance, if a bag is 0.5 meters wide, use 0.6 m of film to create this 0.5 m seam. The extra 0.1 m of film 38 will wrinkle up and be able to be gripped by user and peeled open.

As discussed above, the attachment of the gripping means can be either by welding or by adhesive bonding. Welding can be advantageous in that no extraneous substances potentially migrating through the film are added. Adhesive bonding is however also possible, if an adhesive without migrating cytotoxic components is used or if the gripping means is attached immediately before cultivation.

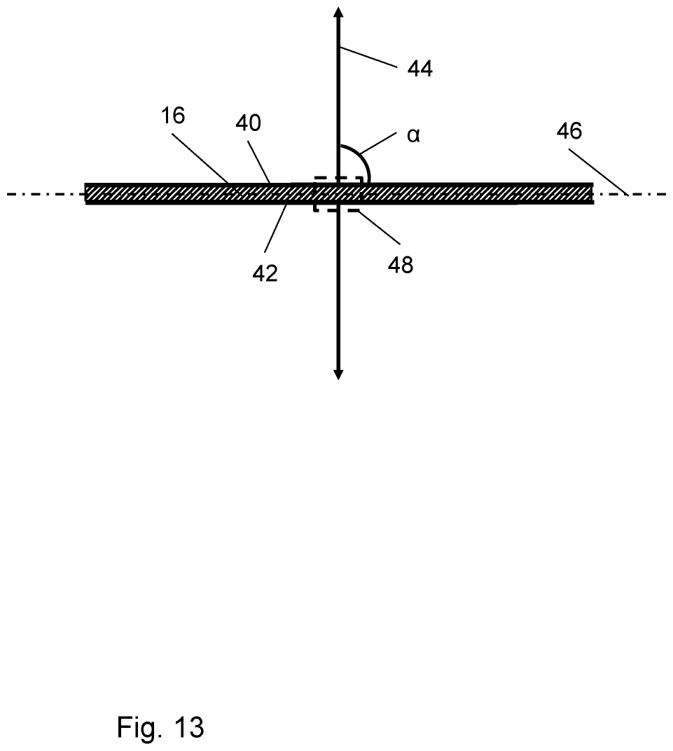

With the gripping means it is possible to pull apart both sides 40,42 of a frangible weld seam 16 in a direction 44 essentially perpendicular to a length axis 46 of the seam, concentrating the forces to a small area 48 (FIG. 13). This is much easier than trying to open the seam e.g. by compressing a cultivation compartment to create an overpressure bursting the seam. An experiment was carried out with Bioclear 10 and 11 films (GE Healthcare), keeping the welding temperature constant at 180° C. and the pressure at 80 psi. Durable weld seams were obtained with 14 s welding time, while frangible weld seams were achieved when the welding time was decreased to 1 s. These frangible weld seams could be opened by perpendicular pulling but not by compartment compression bursting. Further, pulling apart a specific pair of gripping means allows the opening of only the intended frangible seam without any risk of damage to other frangible seams and consequential accidental leakage into adjacent cultivation compartments.

In some embodiments, the bag further includes one or more sensors 50 adapted to measure at least one property in at least one cultivation compartment. Suitably, the bag may include a viable cell density (VCD) sensor. This can e.g. be an inline biomass sensor, e.g. as described in U.S. Pat. No. 8,180,575 or WO 2010/010313A2, which are hereby incorporated by reference in their entireties. Examples of commercially available VCD sensors include Incyte™ (Hamilton) and Futura™ (Aber Instruments Ltd).

In certain embodiments, the bag is adapted to be attached to a rocking or otherwise moving table 52 platform for agitation, either directly or via a tray removably attached to the table. The table can rock back and forth around an axis 54, e.g. placed somewhat below the table. Rocking table platforms suitable for this purpose are described e.g. in U.S. Pat. No. 6,190,913, which is hereby incorporated by reference in its entirety, and are commercially available as WAVE Bioreactor™ from GE Healthcare Bio-Sciences. Table platforms moving in other modes than rocking around a single axis are disclosed e.g. in US20050063247 (table with a vertically pivotable flap), US20080160597 (movement around two parallel axes), US20090233334 (orbital movement) and US20160215249 (gyrating movement), all of which are hereby incorporated by reference in their entireties. These modes are also possible to use with the bags. If the bag is attached to the table or tray, the gripping means 20 on the bottom wall film can be attached to the table/tray. The frangible weld seam can then be opened simply by pulling at the gripping means on the top wall film, either manually or by a mechanical seam opener 56. The attachment of the gripping means to the table/tray may be accomplished by insertion of a (male) gripping means into a corresponding (female) receiver or socket in the table/tray. The receiver/socket can suitably be recessed in the table/tray so as not to have any protruding parts that may risk damaging the bag.

In some embodiments, the bag is supplied presterilized, such as by radiation sterilization or steaming/autoclaving. Radiation sterilization can be accomplished e.g. by gamma or electron beam irradiation. Suitably, all liquid-contact materials are selected to be radiation-stable and to give low levels of leachables also after irradiation. All materials can e.g. be of USP VI quality.

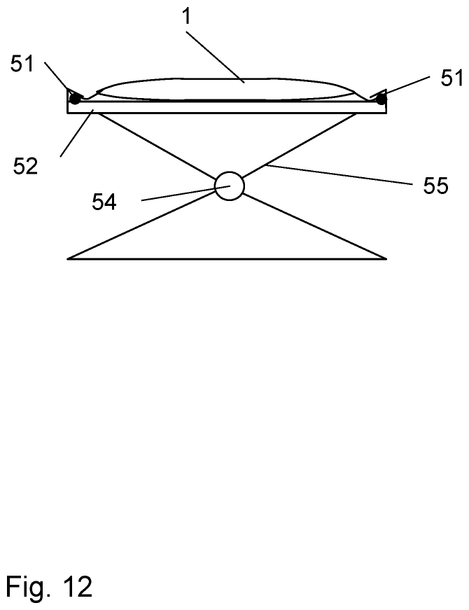

In a second aspect, illustrated by FIGS. 1 and 12, the invention discloses a bioreactor including the flexible plastic bag 1 as discussed above, attached to a moving/rocking table 52 platform for agitation of the bag. The bag can be directly attached to the moving/rocking table or it can be attached via a tray, removably attached to the table. The attachment can e.g. be achieved with rigid rods 51 inserted in pockets at the bag ends and clamped to the tray/table. Suitably, at least one gripping means 20 attached to the outside of said bottom wall film can then be attached to the moving/rocking table platform as discussed above. The rocking table platform can suitably be adapted to rock back and forth around at least one axis 54. The rocking mechanism and the support 55 for the tray/table are described in detail in U.S. Pat. No. 6,190,913 and V Singh: Cytotechnology 30(1-3), 149-158 (1999). The bioreactor may further include a cell culture in at least one of the cultivation compartments. The table/tray may be equipped with a temperature control (heating) surface in direct contact with the bag. It may further be equipped with sensor connectors in electrical contact with at least one bag. At least one of the cultivation compartments may be connected to a gas supply via a gas inlet and a sterile filter.

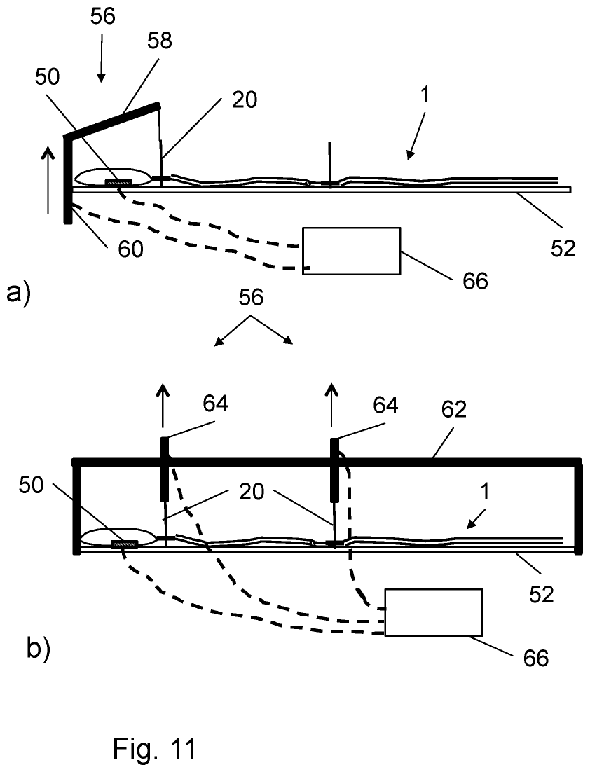

In certain embodiments the bioreactor further includes a seam opener 56, capable of pulling a gripping means 20 attached to the outside of the top wall film to open a specific frangible weld seam. The seam opener can e.g. be connected to a sensor 50 and adapted to open the frangible seam upon receiving a signal from the sensor. In particular, if the sensor is a viable cell density sensor, the seam opener can be adapted to open the frangible seam once a predetermined viable cell density has been reached. The seam opener is arranged to engage a gripping means on the top wall film and to pull the frangible weld seam open in a direction essentially perpendicular to the seam. It can e.g. be a hook or a clamp attached to a solenoid actuator, a vertically movable wire, cantilever, bar or rod etc. The movement can be provided by a motor or piston, optionally via e.g. a cantilever, bar or wire/pulley arrangement. Two illustrative examples are shown in FIG. 11: a) a cantilever beam 58 raised by an actuator 60 attached to the table/tray and b) an overhead beam 62 with two individually adressable actuators 64. As shown in FIG. 11, the bag may include a sensor 50, signally connected to a control unit 66 which is adapted to actuate the seam opener/actuator 56;60;64 once a predetermined signal level is received from the sensor. The seam opener/actuator may further include a force or position sensor adapted to stop the pulling movement once a force or position indicative of complete opening of the frangible weld seam has been achieved.

In a third aspect, the invention discloses a method for cultivation of cells. The method includes the steps of:

a) providing a flexible plastic bag 1 for cultivation of cells, including:

a top wall film 2 and a bottom wall film 4, each having an inside and an outside, sealed to each other inside-to-inside by durable weld seams 12, optionally via one or more side wall films, to form a bag with an inner volume 8 delimited by the durable weld seams;

one or more ports 14 through the top and/or bottom wall film for introduction and withdrawal of fluids;

one or more frangible weld seams 16 joining the insides of said top and bottom wall films, dividing the inner volume into a plurality of cultivation compartments 18;

b) introducing culture media and cells in a first cultivation compartment 18a;

c) cultivating the cells in the first cultivation compartment to provide a cell culture;

d) breaking a frangible weld seam 16 between the first cultivation compartment and an adjacent second cultivation compartment 18b by pulling apart the top and bottom wall films over the frangible weld seam in a direction 44 essentially perpendicular to a length axis 46 of the frangible weld seam 16, so as to combine the first 18a and second 18b cultivation compartments into a larger cultivation compartment. Suitably, the frangible weld seam is completely opened, to avoid any stagnant zones behind residual unopened parts of the seam. If a mechanical seam opener is used, this can involve pulling until a predetermined force level or opener position is reached, which is indicative of complete opening. The pulling movement can then be stopped to avoid any damage to the bag.

e) cultivating the cells in the larger cultivation compartment.

The method may optionally further include the steps of:

f) breaking a frangible weld seam between the larger cultivation compartment and an adjacent third cultivation compartment 18c by pulling apart the top and bottom wall films over the frangible weld seam in a direction essentially perpendicular to the frangible weld seam, so as to combine the first, second and third cultivation compartments into an ultimate cultivation compartment. The pulling direction 44 in steps d) and/or f) can e.g. be at an angle α of 60-90 degrees, such as 75-90 or 80-90 degrees, relative to a length axis 46 of the frangible weld seam (a local length axis if the seam is curved). It is of course also possible to join more cultivation compartments into one ultimate cultivation compartment, e.g. 4 or 5 cultivation compartments.

g) cultivating the cells in the ultimate cultivation compartment.

The cultivations in the cultivation compartments can be made using methods well known in the art and described e.g. in V Singh: Cytotechnology 30(1-3), 149-158 (1999) or Clincke et al., Biotechnol. Prog., 2013, Vol. 29, No. 3. Air or other gases may be supplied via gas inlets and excess air/gas together with gaseous metabolites (e.g. carbon dioxide) may be vented via gas outlets. The cultivation in the first compartment may be continued until a predetermined viable cell density (VCD) is reached, e.g. 1.0×10⁵, 2.0×10⁵, 5.0×10⁵ or 1.0×10⁶ viable cells/ml. When the predetermined VCD has been reached, steps d) and further may be initiated. The VCD can be measured with an inline sensor, as discussed above, or it can be measured off-line or at-line using e.g. the Cytell™ cell imaging system (GE Healthcare Life Sciences) with a cell viability staining kit.

In some embodiments, step g) and/or e) may be conducted in perfusion mode, i.e. by conveying at least part of the culture to a filter where a filtrate is removed and conveying the cells back to the culture and replacing the removed filtrate with fresh culture medium. This allows for further increases in the VCD.

In step a), the bag may be the flexible plastic bag 1 as discussed previously, in which case step d) may include pulling apart the gripping means 20 on the top and bottom wall films adjacent the frangible weld seam. Alternatively, the bag can have one or more frangible weld seams joining the insides of the top and bottom wall films, dividing the inner volume into a plurality of cultivation compartments, with no specific gripping means on one or both of the top and bottom films. In this case, step d) can involve gripping the top and/or bottom film with one or more vacuum suction cups, suction plates, adhesive plates (e.g. a plate covered with double-sided adhesive tape) or similar to pull apart the top and bottom films.

Step c) may include measuring a property of the cell culture with a sensor 50 and, when this property has reached a predetermined value, initiating step d). Suitably the property can be the viable cell density of the cell culture. In this case, the bioreactor may include a control unit 66 (e.g. a computer or PLC), connected to the sensor 50 and a seam opener 56 and programmed to determine if the predetermined value has been reached and arranged to activate a seam opener to perform step d) (FIG. 11).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. All patents and patent applications mentioned in the text are hereby incorporated by reference in their entireties as if individually incorporated.

1. A flexible plastic bag for cultivation of cells, comprising:
a top wall film and a bottom wall film, each having an inside and an outside, sealed to each other inside-to-inside by durable weld seams, optionally via one or more side wall films, to form a bag with an inner volume delimited by said durable weld seams;
one or more ports for introduction and withdrawal of fluids to or from the inner volume;
one or more frangible weld seams joining the insides of said top and bottom wall films, dividing said inner volume into a plurality of cultivation compartments; and
one or more gripping means affixed to said top and bottom wall films, adjacent each of said frangible weld seams and adapted to break a specific frangible weld seam by pulling apart the gripping means on the top and bottom wall films adjacent said specific frangible weld seam.

2. The bag of claim 1, wherein said gripping means comprise one or more of: a loop of film, a tab of film, a hook, a gripping bar, a handle, a port or a fold of the top or bottom film.

3. The bag of claim 1, comprising at least three cultivation compartments.

4. The bag of claim 1, wherein the top and bottom wall films are directly sealed to each other along their edges by durable weld seams.

5. The bag of claim 1, further comprising one or more sensors ow adapted to measure at least one property in at least one cultivation compartment.

6. The bag of claim 5, wherein at least one of said sensors is a viable cell density sensor.

7. The bag of claim 1, adapted to be attached to a moving table platform such as a rocking table platform, for agitation.

8. The bag of claim 1, supplied presterilized, such as by radiation sterilization.

9. The bag of claim 1, comprising a cell culture in at least one of said cultivation compartments.

10. The bag of claim 1, further comprising one or more cultivation media compartments delimited by frangible weld seams.

11. A bioreactor comprising the flexible plastic bag of claim 1, attached to a moving table platform for agitation of said bag.

12. The bioreactor of claim 11, wherein said moving table platform is a rocking table platform.

13. The bioreactor of claim 11, wherein at least one gripping means attached to said bottom wall film is attached to said moving table platform.

14. The bioreactor of claim 13, further comprising a seam opener, capable of pulling a gripping means attached to the outside of said top wall film to open a specific frangible weld seam.

15. The bioreactor of claim 14, wherein said seam opener is configured to pull until said specific frangible weld seam is completely opened and then stop pulling to avoid any damage to the bag.

16. The bioreactor of claim 14, wherein said seam opener is connected to a sensor and adapted to open said frangible seam upon receiving a signal from said sensor.

17. The bioreactor of claim 16, wherein said sensor is a viable cell density sensor and said seam opener is adapted to open said frangible seam once a predetermined viable cell density has been reached.

18. A method for cultivation of cells, comprising the steps of:
a) providing a flexible plastic bag for cultivation of cells, comprising: a top wall film and a bottom wall film, each having an inside and an outside, sealed to each other inside-to-inside by durable weld seams, optionally via one or more side wall films, to form a bag with an inner volume delimited by said durable weld seams; one or more ports for introduction and withdrawal of fluids to or from the inner volume; one or more frangible weld seams joining the insides of said top and bottom wall films, dividing said inner volume into a plurality of cultivation compartments;
b) introducing culture media and cells in at least one of said cultivation compartments;
c) cultivating the cells in said cultivation compartment to provide a cell culture;
d) breaking a frangible weld seam between said cultivation compartment and an adjacent cultivation compartment by pulling apart said top and bottom wall films over said frangible weld seam in a direction essentially perpendicular to said frangible weld seam, so as to combine the two cultivation compartments into a larger cultivation compartment;
e) cultivating the cells in said larger cultivation compartment.

19. The method of claim 18, wherein step a) includes providing a flexible plastic bag that comprises:
a top wall film and a bottom wall film, each having an inside and an outside, sealed to each other inside-to-inside by durable weld seams, optionally via one or more side wall films, to form a bag with an inner volume delimited by said durable weld seams;
one or more ports for introduction and withdrawal of fluids to or from the inner volume;
one or more frangible weld seams joining the insides of said top and bottom wall films, dividing said inner volume into a plurality of cultivation compartments; and
one or more gripping means affixed to said top and bottom wall films, adjacent each of said frangible weld seams and adapted to break a specific frangible weld seam by pulling apart the gripping means on the top and bottom wall films adjacent said specific frangible weld seam.

20. The method of claim 19, wherein step d) comprises pulling apart the gripping means on the top and bottom wall films adjacent said frangible weld seam.

21. The method of claim 18, wherein step c) comprises measuring a property of the cell culture with a sensor and, when said property has reached a predetermined value, initiating step d).

22. The method of claim 21, wherein said property is a viable cell density of the cell culture.

23. The method of claim 21, wherein a control unit is communicatively coupled to said sensor and a seam opener and said control unit is programmed to activate the seam opener once said property has reached the predetermined value, such that the seam opener performs step d).
